(19) United States
(12) Patent Application Publication
(10) Pub. No.: US 2005/0191282 A1
(43) Pub. Date: Sep. 1, 2005

(54) Method of cryopreserving pancreatic islet cells

(76) Inventor: Emmanuel C. Opara, Chicago, IL (US)

Correspondence Address: FLOYD B. CAROTHERS;CAROTHERS AND CAROTHERS, 445 FORT PITT BOULEVARD, SUITE 500, PITTSBURGH, PA, 15219, US

(21) Appl. No.: 11/069,344

(22) Filed: Feb. 28, 2005

Related U.S. Application Data
(60) Provisional application No. 60/549,046, filed on Mar. 1, 2004.

(51) Int. Cl. A01N 1/00; A61K 48/00; C12N 5/08

(52) U.S. Cl. 424/93.7; 435/1.1; 435/366

(57) ABSTRACT

Isolated pancreatic islet cells are cultured and then cyropreserved in a polyvinyl pyrrolidone medium.

### FIELD OF THE INVENTION

The present invention relates to methods of treating isolated pancreatic cells, and more particularly to cryopreservation of such cells.

### BACKGROUND OF THE INVENTION

Glycemic control in diabetes has been sown to delay the onset of, and slow the progression of, associated pathological complications. However, achieving adequate glycemic control using insulin therapy can be difficult. One alternative to insulin therapy is the transplantation of functioning pancreatic islet cells to diabetic subjects, to provide biological insulin replacement.

Approximately one percent of the volume of the human pancreas is made up of islets of Langerhans (hereinafter “islets”), which are scattered throughout the exocrine pancreas. Each islet comprises insulin producing beta cells as well as glucagon containing alpha cells, somatostatin secreting delta cells, and pancreatic polypeptide containing cells (PP-cells). The majority of islet cells are insulin-producing beta cells.

The introduction of the glucocorticoid-free immunosuppression protocol (Shapiro et al, N. Engl. J. Med. 343:230-238 (2000)), which has resulted in insulin independence in 80% of islet transplant recipients at one year (Ryan et al, Diabetes 51:2148-2157 (2002)), has provided convincing evidence that islet transplantation is a viable treatment option for patients with Type 1 diabetes. However, there are still major obstacles to routine use of islet transplantation to treat diabetic patients. These include the severe shortage of human islets and the need to use immunosuppressive drugs (Biancone and Ricordi, Cell Transplant 11:309-311 (2002)). Also, the process of isolating, purifying and transplanting islets at a stretch is labor intensive and difficult for routine treatment. It has been suggested that effective cryopreservation techniques to pre-store islets would enhance routine use of islet transplants even across distant transplant centers, and address the problem of shortage since islets isolated from different donors would be pooled to obtain sufficient islet mass for transplantation (Brunicardi and Mullen, Pancreas 9:281-290 (1994)). Other advantageous effects of cryopreservation islets prior to transplantation include reduction of the immunogenicity of islets (Cattral M S et al, Transplantation 55:159-163 (1993); Letellier C et al, Vox Sang 61:90-95 (1991); Taylor M J et al, Diabetes Res. 5:99-103 (1987); Miyamoto Brunicardi, Transplant Proc. 27-3406-3408 (1995)), and the possibility of tissue matching for HLA phenotypes (Langer S et al, J. Mod. Med. 77:172-174 (1999)).

Dimethylsulfoxide (DMSO) is the most frequently used cryoprotectant in islet cell cryopreservation (Langer S et al, J. Mod. Med. 77:172-174 (1999); Tze W J et al, Metabolism 39:719-723 (1990); Marchetti P et al, Transplantation 57:340-346 (1994); Janjic et al, Pancreas 13:166-172 (1996); Charles K et al, Cell Transplant 9:33-38 (2000); Miyamoto M et al, Transplant Proc. 27:3406-3408 (1995)). However, it is known that the use of DMSO in cryopreservation results in about 30-50% loss in the number and function of islets after thawing (Rich S J et al, Cryobiology 30:407-412 (1993); Rich S J et al, Transplant Proc. 26:823-824 (1994)). Some investigators have reported that the deleterious effects of DMSO can be diminished by the incorporation antioxidants into the cryopreservation medium (Janjic D et al, Pancreas 13:166-172 (1996)). Other investigators have explored the use of other compounds, such as glycerol, ethylene glycol, and polyethylene glycol (PEG), as an alternative to DMSO in islet cryopreservation (Taylor M J et al, Cryobiology 20:185-204 (1983); Korbutt G S et al, Transplantation 64:1065-1070 (1997)). Nevertheless, an optimal procedure for islet cell cryopreservation and the biophysical basis for the protective role of the different cryoprotectants in islet cell freezing remain uncertain.

All compounds used as cryoprotectants are preferentially excluded from the hydration shells of proteins at low temperatures, and this exclusion leads to stabilization. On the other hand, at higher temperatures, these substances interact hydrophobically with proteins and thus act as protein destabilizers and denaturants (Arakawa T et al, Cryobiology 27:401-415 (1990)). Thus, when low molecular weight compounds such as DMSO (MW=0.078 kD) and glycerol (MW=0.092 kD) are used as cryoprotectants, they permeate the cell and may stabilize intracellular proteins during cooling. However, during thawing of frozen cells at higher temperatures, significant destabilization of intracellular proteins would occur with concomitant perturbation of the cytoskeletal architecture, transmembrane ionic pumps, and the integrity of the cell (Arakawa et al, Cryobiology 27:401-415 (1990)).

Other investigators have also shown that the use of DMSO as a cryoprotectant during cryopreservation of islets resulted in significant loss of viable islets and impaired islet function that ranged from 30-50% compared to freshly isolated islets (Rich et al, Cryobiology 30:407-412 (1993), Rich et al, Transplant Proc. 26:823-824 (1994)). In another study, DMSO was compared with glycerol as cryoprotectants for frozen islets. It was found that cryopreservation with these two small-molecular-weight molecules resulted in tissue damage and/or alteration of islet function (Taylor et al, Cryobiology 20:185-204 (1983)). The deleterious effects of DMSO and glycerol as cryoprotectants have also been reported in studies with other cell types. In one study, it was found that PEG was superior to glycerol in reducing formation and the preservation of total adenine nucleotide in cardiac tissues (Banker M C et al, Cryobiology 29:87-94 (1992)). Another study showed that cryopreservation of oocytes with DMSO yielded variable results that were improved by the addition of PEG to the preservation medium (Oneil et al, Cryobiology 34:295-301 (1997)). These observations are consistent with data in the present study, which show that islet recovery and function after cryopreservation with PEG is superior to the use of DMSO and glycerol as cryoprotecants.

The inventor's existing U.S. Pat. No. 6,303,355, issued Oct. 16, 2001, for METHOD OF CULTURING, CYROPRESERVING AND ENCAPSULATING PANCREATIC ISLET CELLS describes procedures to store pig islets by culture or freezing prior to encapsulation for use in transportation. The procedures involve the use of proprietary media to diminish damage to the cells during the processes of isolation, culture and freezing of the cells. It is an object of the present invention to alleviate the need for use of such proprietary media in the isolation, culture or freezing of the cells.

### SUMMARY OF THE INVENTION

The present invention provides a method of treating isolated pancreatic islet cells by first culturing the isolated pancreatic islet cells and then cyropreserving the cultured cells in a polyvinyl pyrrolidone (PVP) medium.

PVP has been used as a cryoprotectant for other cell types (Loretz L J et al, Xenobiotica 19:489-498 (1989), Bakaltcheva I et al, Cryobiology 40:283-293 (2000)), however, it has not been hitherto used for cryoprotection in islet cell freezing. It is noteworthy that previous studies with PVP as a cryoprotectant have all been performed using single cells. An islet is a composite tissue of cells that are loosely bound together and need each other for normal function. This unique structure of islets makes them particularly fragile and sensitive to cellular perturbation. It is therefore exciting that PVP is able to protect islets from cryoinjury without interfering with function, which is in contrast to other compounds, such as DMSO, that have been found as suitable for cryoprotection of single cells, but not islets.

In summary the present establishes that PVP is as effective as PEG and that a higher percentage of intact islets is obtained when islets are cryopreserved with PEG and PVP than when DMSO and glycerol are used as cryoprotectants. It was also found that while the function of islets cryopreserved with DMSO and glycerol is impaired compared to freshly isolated islets, there is no loss of function when islets are cryopreserved with either PEG or PVP.

### BRIEF DESCRIPTION OF THE DRAWINGS

FIG. 1 graphs glucose stimulated insulin secretion in fresh isolated islets. Following effluent basal sample collection after preperifusion, the glucose concentration in the perifusate was raised to 16.7 mM with continuous effluent sample collection on ice. All samples were stored frozen at minus 20° C. until radio radioimmunoassay for insulin. Data represent mean±SEM.

FIG. 2 graphs glucose stimulation of insulin secretion in islets cryopreserved with different cryoprotectants. After one week of cryopreservation, islets were thawed and cultured overnight before being tested for response to 16.7 mM glucose stimulation. The 16.7 mM glucose stimulated insulin secretion was assessed by normalizing data as percent basal insulin secretion for each group of islets. Data represent mean±SEM.

### DETAILED DESCRIPTION OF A PREFERRED EMBODIMENT

The present invention is based on the hypothesis that the low molecular weight (MW) compounds, DMSO and glycerol, permeate the cell and interact hydrophobically with intracellular proteins thereby perturbing the cyto-skeletal architecture of frozen cells, and diminishing islet cell integrity and function. Isolated rat islets were cultured overnight (16-24 hr) at 37° C., in RPMI medium supplemented with 10% fetal calf serum and 1% mixture of Penicillin/Streptomycin. Using a programmable temperature controller, samples of pre-counted islets were then frozen under liquid nitrogen, in the presence of either 2M DMSO (MW=0.078 kD), 3M glycerol (MW=0.092 kD), 5% polyethylene glycol (PEG, MW=20 kD), or 10% polyvinylpyrrolidone (PVP, MW=40 kD), and stored at −80° C. for 1 week. Following thawing, intact islet recovery was determined by islet counting after Dithizone staining. Islet function was assessed by determination of glucose-stimulated insulin secretion in perifusion experiments with Krebs-Ringer bicarbonate buffer, pH 7.4, containing either basal (3.3 mM) or high (16.7 mM) glucose concentrations. After thawing samples of frozen islets, the mean±SEM % intact islet recovery was higher with PVP compared to DMSO (82±4.6 versus 62.7±3.1% respectively, p<0.005, n=9). Furthermore, the glucose stimulation index of insulin secretion in islet samples frozen with PEG and PVP was comparable to that of freshly isolated islets, in contrast to DMSO and Glycerol. There was no significant difference in intact islet recovery and function between samples frozen with PVP and those frozen with PEG. Samples frozen with DMSO and Glycerol had poor results similar to each other in islet recovery and function. These data show that PVP is a new cryoprotectant for islet cell freezing.

It has been shown that high molecular weight cryoprotectants such as polyethylene glycol (MW=20 kD) increase the viscosity of the medium at low temperatures and inhibit ice crystal growth during cooling or re-warming, without permeating the cell (O'Neil, L et al, Cryobiology 34:295-301 (1997)). Based on this physical property of high molecular weight cryoprotectants, the purpose of the present study was to determine the efficacy of polyvinyl pyrrolidone (PVP) in islet cell cryopreservation. Although PVP is a high molecular weight compound (MW=40 kD) that has been used as a cryoprotectant for other cell types (Loretz L J et al, Xenobiotica 19:489-498 (1989), Bakaltcheva I et al, Cryobiology 40:283-293 (2000)), it has not been hitherto used for cryoprotection in islet cell freezing. Furthermore, in order to establish the relationship between the efficacy of the cryoprotectants and their molecular sizes, the efficacy of other cryoprotectants having different molecular weights was also tested, and used in islets cell freezing.

As used herein, culture refers to the maintenance or growth of cells on or in a suitable nutrient medium, after removal of the cells from the body. Suitable nutrient culture media are readily available commercially, and will be apparent to those skilled in art given the cell type to be cultured.

### EXAMPLE

### Materials and Methods

Animals and Supplies:

Adult female Sprague-Dawley rats were obtained from Charles River. The rats were housed in stainless steel cages and maintained at a temperature of approximately 37° C. with a 12:12 hour light/dark cycle. They were placed on a diet consisting of laboratory chow diet (Purina, St. Louis, Mo.). Collagenase Type P was obtained from Boehringer Mannheim Co. (Indianapolis, Ind.). RPMI 1640 was obtained from Gibco, Grand Island, N.Y. Highly purified bovine serum albumin (BSA, fraction V) free of fatty acids and insulin-like activity, sodium alginate and all chemicals for cryopreservation and buffer solutions were purchased from Sigma Chemical Company (St. Louis, Mo.). Crystalline pork insulin was a gift from Dr. Ronald Chance, Eli Lilly (Indianapolis, Ind.) and monoiodinated ¹²⁵I-insulin was obtained from New England Nuclear (Boston, Mass.). Cryotubes (4 mL) were purchased from VWR.

Isolation and Culture of Islets:

Using sterile techniques, islets were isolated by the collagenase digestion of pancreatic tissue as described by Lacy P E et al, Diabetes 16:35-39 (1967). After separating the undigested tissue from isolated islets followed by washing of the islets, intact islets were selected by handpicking with a pipette under a dissecting microscope. Some islets were encapsulated and tested immediately for their response to glucose stimulation and others were tested after cryopreservation followed by an overnight (18-24 hour) culture after thawing. Also, the procedure adopted for the cryopreservation of isolated islets requires an overnight culture prior to freezing, as outlined below. Following handpicking of islets, they were placed in sterile culture dishes containing RPMI 1640, supplemented with 10% fetal calf serum and 1% mixture of antibiotics (penicillin/streptomycin) and cultured overnight (18-24 hours) prior to storage by freezing. The culture dishes were incubated at 37° C., in an atmosphere of humidified 95% air and 5% CO₂, as previously described (Charles et al, Cell Transplant 9:33-38 (2000)).

Cryopreservation and Thawing of Islets:

Isolated islets were stored by cryopreservation using a modification of the procedure that we have previously described (Charles et al, Cell Transplant 9:33-38 (2000)). Briefly, following purification by Ficoll gradient separation and handpicking, the islets were first cultured overnight (within an approximate range of 12 to 36 hours) prior to being frozen under liquid nitrogen using cryotubes containing Medium 199 supplemented with 4% Penicillin/Streptomycin, 10% fetal calf serum, 0.7 mM L-glutamine and 25 mM HEPES, and one of four cryoprotectants, namely: DMSO, glycerol, PEG, and PVP. The final concentrations of the cryoprotectants used were chosen from initial dose response studies, and the medium was adjusted to pH 7.4. For the islets frozen with DMSO and glycerol as cryoprotectants, there was a stepwise addition of the cryoprotectant medium over a period of 30 minutes in order to minimize the osmotic stress to the islets. A pre-counted number of islets were suspended in the medium and were first equilibrated with either 0.67M, followed by 1M, then 1.5M DMSO, or 2M glycerol for 5 minutes in each case. Then the concentration was finally increased to 2M DMSO and 3M glycerol for 15 minutes before freezing. When the other cryoprotectants were used, the islets were equilibrated with either 5% PEG or 10% PVP for 5 minutes before freezing with a programmable temperature controller (Gordiner Electronics, model 7009). The cryoptubes were placed in the freezing chamber, supercooled to −7° C. and nucleated. They were subsequently frozen at the rates of −3° C./minute to −40° C., and −5° C./minute to −100° C.

The frozen islets were stored at −80° C. for at least one week, and when needed, were thawed by direct incubation in a water-bath maintained at 37° C. (Charles et al, Cell Transplant 9:33-38 (2000)). To prevent osmotic shock and enhance islet survival, the stepwise dilution protocol described by Lakey J R T et al, Cell Transplant 6:163-172 (1997) was used to process the thawed samples. One mL of either 0.75 M sucrose (for glycerol and DMSO preserved samples) or the supplemented culture medium (for PEG and PVP samples) was added to each cryotube containing the cryopreserved islets and kept on ice for 30 minutes. The contents of the cryotubes were then transferred to 15 mL conical centrifuge tubes before 1, 2, and 4 mL of the culture medium was added sequentially at 5-minute intervals at room temperature. The islets were separated by gentle centrifugation at 1000 rpm for 5 minutes and kept in a humidified 5% CO₂ incubator for 24 hours prior to determination of recovery after Dithizone staining, and testing of function by glucose stimulation of insulin secretion by perifused islets.

Perifusion of Islets:

Groups of ten islets were placed in flow-through miniature perifusion chambers for testing of islet response to glucose stimulation as previously described (Charles et al, Cell Transplant 9:33-38 (2000)). Islets were pre-perifused for 1 hour at 37° C. with a modified Krebs-Ringer bicarbonate buffer (Opara E C et al, Endocrinology 130:657-662 (1992)), which contained 3.3 mM (60 mg/dL) glucose and was maintained at pH 7.4 by continuous gassing with 95%/5% air/CO₂. After pre-perifusion, basal effluent samples were collected on ice at 5-minute intervals for 20 minutes prior to raising the glucose concentration in the perifusate for 20-30 minutes with effluent sample collection. Following the high glucose stimulation, the islets were returned to a basal glucose perifusion for 20 minutes and samples were also collected. Solutions were changed using a system of stopcocks and all samples were stored frozen at −20° C. until assayed for insulin content (Herbert V. et al, J. Clin. Endocrinol Metab. 25:1375-1384 (1965)).

Assessment of Insulin Secretion and Data Analysis:

The data were assessed as mean±standard error (SEM) rate of insulin secretion in pg/10 islets/min. For data requiring multiple comparisons, statistical evaluation was performed using an analysis of variance (ANOVA) computer program and depending upon the outcome of ANOVA, the Bonferroni correction was performed to assess the significance of differences among multiple means. A student's t-test was used to compare the difference between means of two groups of data. In all cases a value of p<0.05 was accepted as significant.

### Results

Effect of High Glucose on Insulin Secretion by Freshly Isolated Islets:

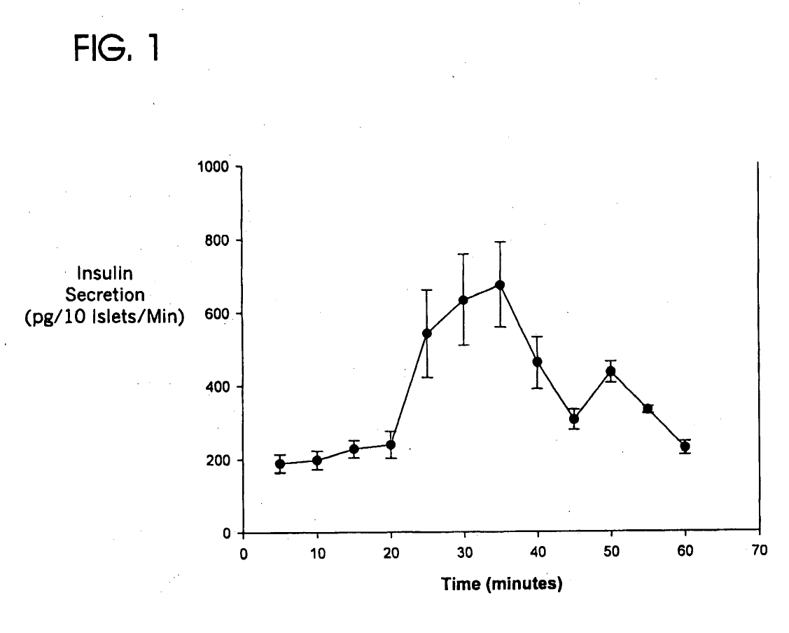

Insulin secretion in response to 16.7 mM glucose stimulation in freshly isolated rat islets is illustrated in FIG. 1, which shows a stimulation index greater than 3 (p<0.01, n=4).

Effects of Cryoprotectants on Recovery and Function of Frozen Islets:

As shown Table 1, the percent recovery of intact islets after thawing samples of frozen islets was higher with PVP had PEG compared to DMSO and glycerol (p<0.05). Consequently, there was severe fragmentation of the islets after thawing samples frozen with DMSO and glycerol as cryoprotectants (Table 1). There was no significant difference in the recovery of islets from the PVP and PEG-cryopreserved samples, and the Dithizone staining of the islets cryopreserved with these two high-molecular-weight cryoprotectants was more intense than those of DMSO and glycerol samples.

TABLE 1
Recovery of intact islets after cryopreservation

| Experiment# | DMSO | GLY | PEG | PVP |
|---|---|---|---|---|
| 1 | 68 | 28 | 50.7 | 90 |
| 2 | 61.7 | 55.6 | 62.7 | 96.7 |
| 3 | 60 | 20 | 68.3 | 95 |
| 4 | 54.7 | 72.5 | 64 | 65 |
| 5 | 78.7 | 28 | 68 | 94 |
| 6 | 55.3 | 80 | 66 | 90 |
| 7 | 70 | 24 | 92 | 76 |
| 8 | 68.1 | 26 | 72 | 64 |
| 9 | 48 | 73.3 | 72.7 | 67 |
| Mean | 62.7 | 45.3 | 68.5 | 82.0 |
| SE | 3.1 | 8.2 | 3.7 | 4.6 |
| Fragment | +++ | +++ | + | + |

Severe = +++
Mild = +

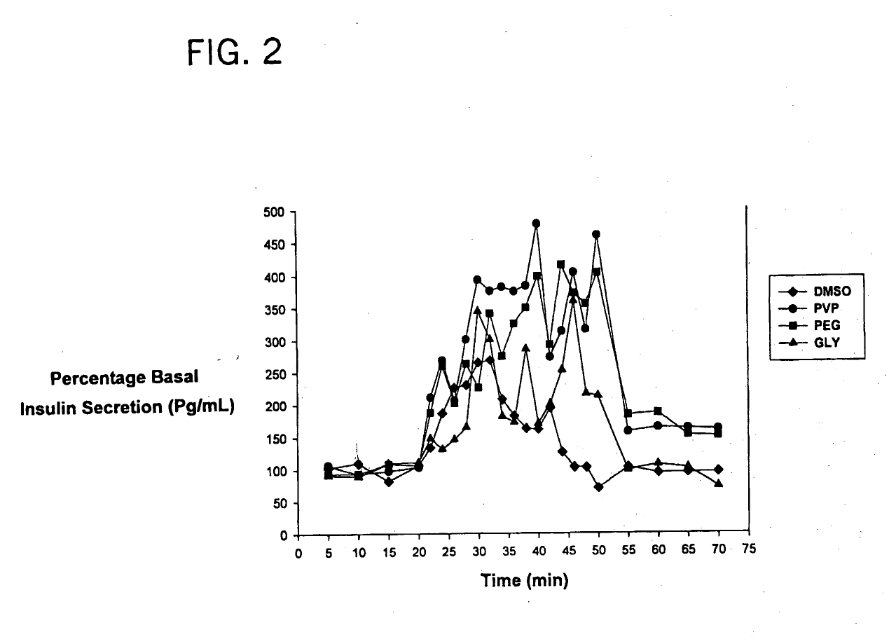

The glucose-stimulation index of insulin secretion by islets recovered after cryopreservation with the different cryoprotectants is shown in FIG. 2. Glucose stimulation of insulin secretion by islets frozen with PVP and PEG as cryoprotectants was higher than that obtained in islets cryopreserved with either DMSO or glycerol (p<0.01). No difference was observed in the glucose stimulation index of islets cryopreserved with PEG and PVP, which was greater than 3, as in the case of the freshly isolated islets in FIG. 1.

### Discussion

The data in the present study show that when samples of isolated islets frozen with high-molecular-weight cryoprotectants are thawed, a higher percent of intact islets are obtained than samples cryopreserved with the small-molecular weight compounds, glycerol and DMSO. The islets recovered after freezing with DMSO and glycerol have a significant loss of function as measured by in vitro stimulation of insulin secretion. These data are consistent with the notion that the small-molecular-weight compounds permeate the cells and perturb the cytoskeletal architecture and function of the cells through hydrophobic interactions with the intracellular proteins (Arakawa et al, Cryobiology 27:401-415 (1990)). It is of interest that among the adversely affected intracellular proteins, are key regulatory enzymes of glycolysis, such as phosphofructokinase (Arakawa et al, Cryobiology 27:401-415 (1990)). It is well-established that glycolysis is the primary event in glucose metabolism which is linked to glucose stimulation of insulin secretion (Meglasson M D & Matschinsky F M, Am. J. Physiol 246 (1pt.1):E1-E13 (1984); Murata T et al, Diabetes 42:1003-1009 (1993)). It is therefore possible that impaired insulin secretion observed after cryopreservation with the small molecular weight cryoprotectants may be linked to their adverse effects on glycolytic enzymes of the beta cells.

The foregoing is illustrative of the present invention, and is not to be construed as limiting thereof. The invention is defined by the following claims, with the equivalence of the claims to be included therein.

1. A method of treating isolated pancreatic islet cells comprising:
(a) culturing said isolated pancreatic islet cells; and
(b) cyropreserving said cultured cells in a polyvinyl pyrrolidone medium.

2. A method according to claim 1, including thawing said cyropreserved cells.

3. A method according to claim 1, wherein said isolated pancratic cells are cultured in the presence of an antioxidant.

4. A method according to claim 3, wherein culturing said isolated pancreatic islet cells is carried out within an approximate time range of about 12 to 36 hours.
